(19) United States
(12) Patent Application Publication
(10) Pub. No.: US 2025/0009299 A1
(43) Pub. Date: Jan. 9, 2025

(54) INSERTION DEVICE, INSERTION SYSTEM AND METHOD FOR INSERTING A MEDICAL DEVICE

(71) Applicant: Roche Diabetes Care, Inc., Indianapolis, IN (US)

(72) Inventor: Michel Alain Jean Cadio, Carmel, IN (US)

(21) Appl. No.: 18/893,490

(22) Filed: Sep. 23, 2024

Related U.S. Application Data
(63) Continuation of application No. PCT/EP2023/057258, filed on Mar. 22, 2023.
(60) Provisional application No. 63/323,190, filed on Mar. 24, 2022.

(30) Foreign Application Priority Data
Apr. 29, 2022 (EP) 22 170 772.2

(51) Int. Cl. A61B 5/00; A61B 5/145

(52) U.S. Cl. CPC A61B 5/6846; A61B 5/14503; A61B 5/1451; A61B 5/14532; A61B 2560/063

(57) ABSTRACT

An insertion device has a guide sleeve at its proximal end that contacts the skin for an insertion process. A cap at the distal end of the device can be pressed to carry out the insertion process while moving in the direction of the proximal end. An inserter sleeve is at least partially within the guide sleeve and/or the cap and has a holder for the medical device, and the inserter sleeve is moveable with respect to the guide sleeve. A retractor is moveable along a movement path and retracts a portion provided with the medical device. An elastic element biases the retractor towards the distal end along the movement path. A retractor control controls the movement of the retractor by blocking or freeing the movement of the retractor along the movement path, and the retractor control is rotatable with respect to the retractor.

### RELATED APPLICATIONS

This application is a continuation of PCT/EP2023/057258, filed Mar. 22, 2023, which claims priority to EP 22 170 772.2, filed Apr. 29, 2022, and also claims priority to U.S. Patent Application Ser. No. 63/323,190, filed Mar. 24, 2022, all of which are hereby incorporated herein by reference.

### BACKGROUND

This disclosure refers to an insertion device for at least partially inserting a medical device such as an analyte sensor.

U.S. Publication No. 2019/0350527 discloses an insertion device in which a needle carrier is twisted during the retraction process.

U.S. Pat. No. 9,522,225 B2 describes a needle inserter device in which the insertion process requires the application of force against a spring which provides the force to retract a needle.

### SUMMARY

This disclosure teaches an insertion device which is easy to use and avoids rotation of parts that contact a needle which may rotate the needle and affect user comfort. This disclosure also teaches an insertion system which comprises such an insertion device and a medical device. This disclosure also relates to a method for inserting a medical device with such an insertion device.

The insertion device is provided in order to at least partially insert a medical device. This medical device may be, or comprise, an analyte sensor such as a glucose sensor.

At least part of the medical device is to be inserted through the skin of a subject. The subject may be a human being such as a patient suffering from diabetes or an animal.

The medical device may be provided for various purposes. It may be provided in order to withdraw samples from the subject, e.g., by providing a cannula; it may be provided in order to provide medication or liquids to the subject (also possibly by a cannula); and/or it may be provided to gain information on the subject by sensors such as an analyte sensor like a glucose sensor.

The insertion device has a proximal end that is to be put in contact with the skin and it has a distal end opposite the proximal end. The device may be arranged such that the user can act on the distal end of the insertion device in order to insert the medical device, while the proximal end is in contact with the skin of the subject, which may be the user or another person or animal.

The size of the insertion device from the distal end to the proximal end may be in the range of 60 to 80 mm such as between 65 and 75 mm. This refers to the size of the insertion device prior to being used for an insertion process. In that state an elastic element is, for example, still pre-loaded, as will be explained further below and/or no force is applied in order to push the cap and/or the guide sleeve towards each other.

This size of the insertion device from the distal end to the proximal end may depend on the size of the medical device. A larger/smaller medical device may have a larger/smaller insertion device. It may also depend on the length of the portion to be retracted or the desired distance between the end of the portion to be retracted from the skin. The smaller the desired distance the smaller the size of the insertion device.

The insertion device comprises a guide sleeve, which is provided at the proximal end of the insertion device and is to be placed in contact with the skin of the subject for the insertion process. This guide sleeve may exert force on the skin of the user in the insertion process which fixates the guide sleeve on the skin and avoids relative movement of the guide sleeve parallel to the surface of the skin in order to allow a safe insertion process.

A cap is provided at the distal end of the insertion device which may be pressed on to carry out at least part of the insertion process, while moving in the direction of the proximal end of the insertion device. The insertion process may also involve a movement of the cap in the opposite direction from the proximal end of the insertion device towards the distal end of the insertion device. This movement may be shorter than the movement in the direction of the distal end towards the proximal end.

The guide sleeve and the cap together may provide a housing of the insertion device and may be the only visible parts or touchable parts by a human user when the insertion device is provided on the skin of a subject.

The insertion device furthermore comprises an inserter sleeve provided at least partly within the guide sleeve and/or the cap and is provided with a holder for the medical device. The inserter sleeve is moveable with respect to the guide sleeve. The inserter sleeve may be adapted to hold the medical device, part of which is to be inserted, and the movement of the inserter sleeve, with respect to the guide sleeve, allows approximating the medical device towards and (partially) through the skin of the user.

The medical device may comprise a part which is to be inserted through the skin of the subject and it may comprise another part which is to be applied on the outside of the skin of the subject, e.g., by an adhesive.

The medical device may, for example, be an analyte sensor which is combined with electronics. This sensor is to be inserted through the skin of the subject with, e.g., electrodes that can measure an analyte concentration such as a glucose concentration in an interstitial fluid of the subject. The medical device may, furthermore, include a (wearable or portable) electronic circuit which may be battery powered that processes information obtained by the inserted part of the medical device such as by the sensor. The electronics may be configured to be attached to the skin of the subject by an adhesive. The adhesive may be provided on an outer surface of a housing that contains the electronics.

The insertion device, furthermore, comprises a retractor which is configured to retract a portion provided with the medical device such as a needle and the retractor is provided moveable along a movement path. A medical device which has a part that is to be inserted, often is inserted with the use of a portion provided with the medical device such as a needle, which itself is only provided for the insertion process and retracted afterwards. The portion provided with the medical device will not be provided with the medical device after the insertion process. It will be disconnected therefrom. The portion provided with the medical device may give stability to the part/sensor that is to be inserted, which itself may not necessarily be rigid for user comfort. After the part/sensor is inserted, the portion/needle can be retracted and the part/sensor remains inserted.

The insertion device, furthermore, comprises an elastic element such a spring, which is provided to bias the retractor towards the distal end along the movement path.

In order to control the movement of the retractor, a retractor control element is provided which may block or free the movement of the retractor along the movement path. The retractor control element itself is moveable with respect to the retractor. In case the retractor control element frees the movement path of the retractor, the elastic element may move the retractor along this freed movement path. Thereby, a rapid retraction of the portion of the medical device, such as a needle to be retracted, is achieved. The movement of the retractor-once freed to move along the movement path—may include a movement beyond the end of a movement path defined by guide portions of the inserter device.

With such a configuration, it is possible to provide an insertion device with a pre-loaded elastic element such that for the insertion process no force against the elastic element, which may provide the movement of the retractor towards the distal end, is necessary.

Furthermore, as the retractor control element is moveable, with respect to the retractor, the motion of the retractor can be confined to the linear movement, avoiding possible rotational movement of the portion that is to be retracted and thereby on the part that is to be inserted. Avoiding rotational movement of the portion to be retracted or the part to be inserted improves the user experience by avoiding a potential source of pain and by avoiding undesired deformation of the part to be inserted such as of the sensor.

The retractor control element may be rotatable into a position in which it blocks the movement of the retractor along the movement path and another position in which it frees the movement of the retractor along the movement path. The use of a rotational movement of the retractor control element allows the device (e.g., extension of the device in a direction perpendicular to the surface of the skin to which it may be applied, i.e., the size measured from the distal to the proximal end of the insertion device) to be relatively small as, for the rotational movement, no additional space for a longitudinal movement inside the insertion device is necessary.

The retractor control element may be provided rotatable by a portion of the guide sleeve which comes into contact with the retractor control element, such as with a protruding portion of the retractor control element upon a relative movement of the retractor control element, with respect to the guide sleeve. As the relative movement of the retractor element, with respect to the guide sleeve, at least to some extent, is a linear movement the portion of the guide sleeve may be provided with a ramp shape, which translates the linear movement into a rotational movement of the retractor control element, as the protruding portion of the retractor control element slides along the portion of the guide sleeve, such as the ramp which has the ramp shape. This allows for a well-defined rotational movement of the retractor control element.

The movement of the retractor is confined to a linear movement in the direction of the proximal end to the distal end, or vice versa. This confinement to a linear movement avoids any rotational movement of the retractor which may cause a rotation of the part that is to be inserted, or that is inserted into the skin of the subject. The confinement to the linear movement is optionally provided by at least one pair of guide portions, the pair of guide portions may comprise of the first guide portion, such as a rail, and cooperate with a second guide portion such as a negative rail, where one of the two guide portions is provided at the inserter sleeve and the other guide portion is provided at the retractor. The guide portions may be provided as straight elements providing a linear guiding. Such guide portions allow for a safe way to define the linear movement of the retractor. In order to provide a stable and wiggle free movement, optionally, there are three such pairs of guide portions provided. They may be provided with the same distance to each other, or at least two of the three pairs may be provided with the same distance with respect to each other. Thereby, unintentional mechanical blocking of the retractor can well be avoided.

The guide portions of the inserter sleeve may provide a portion of the movement path of the retractor. Another portion of the movement path of the retractor may be provided by guide portions of the retractor control element. The movement path of the retractor may be provided by the guide portion of the inserter sleeve and the guide portions of the retractor control element in combination.

The retractor control element may be provided with at least one guide portion which can be brought into alignment with another guide portion such as the above mentioned guide portion of the inserter sleeve in order to free the movement of the retractor along a portion of the movement path and it may block the movement of the retractor along the portion of the movement path in case the guide portion of the retractor control element is not in such alignment. Using the guide portions that define the linear movement of the retractor, also for mechanically allowing or blocking the movement of the retractor along its movement path, provides for a mechanically simple construction allowing for a failsafe design. The movement path may be provided by aligned guide portions of the inserter sleeve and of the retractor control element.

The retractor control element may be provided with at least one blocking protrusion which is provided to interact with the blocking portion of the cap, preventing unintentional rotation of the retractor control element into a position in which it frees the movement of the retractor. This interaction between a blocking protrusion and a blocking portion of the cap allows to well define the moment in which the retractor control element is allowed to free the path of the retractor. Optionally, this interaction between the blocking protrusion and the blocking portion occur when the cap is completely pressed down, i.e., when the cap has reached its most proximal position relative to the skin of the subject. Upon slightly retracting the cap away from this most proximal position towards a more distal position, the interaction may stop, thereby freeing the rotation of the retractor control element towards a position in which it frees the movement of the retractor along the movement path.

With help of this blocking protrusion and the blocking portion it is possible to have the retraction process only to take place once the cap is moved away from the skin of the subject. This provides control to the user of the insertion device in such a way that pushing on the cap towards the skin of the subject, inserts the part that is to be inserted and the movement of the cap away from the skin of the subject initiates the retraction of the portion of the medical device that is to be retracted.

The retractor control element may comprise a locking portion which is in contact with an engagement portion of the cap, which prevents rotation of the retractor control element as long as the cap is positioned in its most distal position. When shipping the device or preparing the device for the insertion process, the cap typically will be in this most distal position. It is optionally provided to prevent at that moment an unintentional retraction movement of the retractor. Therefore, it is provided that the retractor control element may not rotate as long as the cap is in its most distal position. This also helps to improve the results of the insertion device in a drop test in which the device is dropped on a hard surface with the elastic element being pre-biased. Slight mechanical deformations in the drop test do not lead to an unintended retraction process prior to insertion. The contact between the locking portion of the retractor control element and of the engagement portion of the cap is lost once the cap is moved from its most distal position towards its most proximal position. The contact may be established, for example, for the movement of the cap towards its most distal position of five to ten percent of its movement such as a distance in a range between 0.5 to 3 mm.

The retractor control element may be mounted rotatably on the inserter sleeve. By being mounted on the inserter sleeve, the retractor control element is well positioned with respect to the retractor, as the retractor may be provided within and be held and/or be guided by the inserter sleeve.

The movement of the cap with respect to the guide sleeve may be provided such that only a linear movement relative to the guide sleeve is possible by the cap. This allows for an easy to use insertion device as no complex movements of the cap with respect to the guide sleeve are necessary. In order to define this linear movement, (straight) guide portions may be provided on the cap and second guide portions co-operating with the first guide portions may be provided on the guide sleeve. Optionally, there are at least three such pairs of guide portions which allows to define a relative movement without blocking or wobbling of the two parts with respect to each other. For that purpose, it is optionally possible that the guide portions are equally spaced with respect to each other, such as at equally distanced circumferential positions.

The cap may be provided with pushing portions for pushing the inserter sleeve towards the proximal end of the insertion device and optionally also with separation limiting means which limit a separation of the cap away from the inserter sleeve towards the distal end of the insertion device. The pushing portions allow to translate the movement of the cap from its distal position towards the proximal position, also into a movement of the inserter sleeve from a distal position towards a proximal position. This movement of the inserter sleeve from a distal position to a proximal position allows to perform the insertion process.

The separation limiting means avoid that the cap may separate itself from the inserter sleeve in an uncontrolled fashion. The pushing portions together with the separation limiting means may define the allowed relative movement of the cap with respect to the inserter sleeve. This allowed relative movement may be between 1 to 5 mm or between 1 and 3 mm.

The cap, the inserter sleeve, the retractor and the retractor control element as well as the elastic element, may be together moveable from a distal position to a proximal position, at least along a portion of the movement of the cap; this movement takes places for at least a portion without a relative movement of the five parts with respect to each other. Along another portion of the movement of the cap at least the retractor control element may perform a purely rotational movement with respect to the other parts while the other parts move without a relative movement relative to each other.

The elastic element such as the spring may be provided in the pre-loaded state in which the spring is pressed together in order to store the energy for the retraction process. The elastic element may be provided to act between the retractor and the inserter sleeve. This means that the elastic element provides a force between the retractor and the inserter sleeve which may cause the retractor to move relative to the inserter sleeve once the movement path of the retractor is freed by the retractor control element.

The insertion system comprises an insertion device as described above or below and furthermore, it comprises the medical device to be inserted with the insertion device.

In the method for inserting a medical device such as an analyte sensor with an insertion device, as described above or below, the following steps are carried out. First the insertion device is applied to the skin of a subject. Then the cap of the insertion device is pressed towards the skin of the subject, the resulting linear movement of the cap relative to the guide sleeve translates into a rotational movement of the retractor control element relative to the guide sleeve and optionally also relative to the cap and/or the inserter sleeve. The retractor control element frees a movement path of the retractor towards the cap by rotation of the retractor control element. This optionally happens after the cap has slightly retracted in the direction away from the skin of the subject. In this way the insertion process has two clearly defined steps, one in which the cap is moved towards the skin and another one in which the cap is moved away from the skin. The first one leading to the insertion of at least a part of the medical device and the second one leading to the retraction of a portion provided with the medical device to be retracted, such as a needle.

### BRIEF DESCRIPTION OF THE DRAWINGS

The above-mentioned aspects of exemplary embodiments will become more apparent and will be better understood by reference to the following description of the embodiments taken in conjunction with the accompanying drawings, wherein:

FIG. 1a is an exploded view of an insertion device;

FIG. 1b details some parts of the exploded view of FIG. 1a;

FIGS. 2a to FIG. 2c are different views of the inserter sleeve, the retractor and the retractor control element;

FIG. 3a and FIG. 3b are different sectional views of the cap, the guide sleeve and the inserter sleeve;

FIG. 4 is a sectional view of an insertion device;

FIG. 5a and FIG. 5b are different 3D views of the insertion device; and

FIGS. 6a-6e are sectional views of different states of the insertion device during the insertion process.

### DESCRIPTION

The embodiments described below are not intended to be exhaustive or to limit the invention to the precise forms disclosed in the following detailed description. Rather, the embodiments are chosen and described so that others skilled in the art may appreciate and understand the principles and practices of this disclosure.

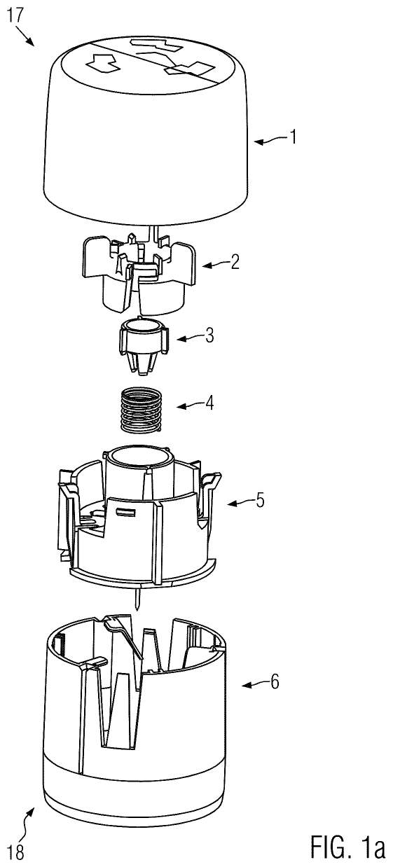

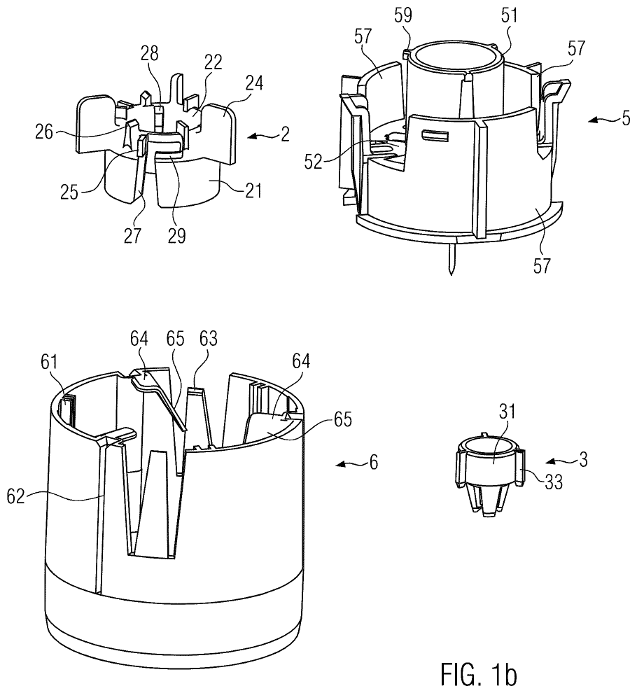

FIG. 1a shows an exploded view of different parts of an embodiment of an insertion device. The insertion device may comprise a cap 1, a retractor control element 2, a retractor 3, an elastic element 4, an inserter sleeve 5 and a guide sleeve 6.

The cap 1 may be provided with an essentially cylindrical outer shape. The side wall of the cap may have a cylindrical or (slightly) conical shape. On the distal end 17 of the insertion device, the cap may have an essentially flat upper surface which serves as a surface on which a user can apply force by hand. The cap 1 may provide an inner volume into which other parts of the insertion device may be accommodated.

The retractor control element 2, the retractor 3, the elastic element 4 and the inserter sleeve 5 will be explained in more detail in the following figures. The guide sleeve 6 is provided with (essentially) cylindrical side wall and is open at its distal and proximal end. The cap 1, together with the guide sleeve 6, provide the housing of the insertion device. They form a joint inner space in which the retractor control element 2, the retractor 3, the spring 4 and the inserter sleeve 5 are provided.

The insertion device has a distal end 17 and a proximal end 18. The proximal end 18 is supposed to be placed in contact with the skin of a subject.

The insertion process may be performed by placing the proximal end 18 of the guide sleeve onto the skin of a subject and by exerting force, e.g., by pressing on the distal top surface of the cap 1 and by moving the cap 1 towards the proximal end 18 or in other words towards the guide sleeve 6. The insertion process is terminated (as will be detailed further below) by the cap 1 being moved slightly in a distal direction (towards the distal end 17) after having been pressed on.

In FIG. 1b some parts shown in FIG. 1a are displayed separately and are provided with some reference numerals used further below. The reference numerals of FIG. 1b appear in other figures as well and FIG. 1b allows for a better understanding of the configuration of the distinct parts shown in the following figures.

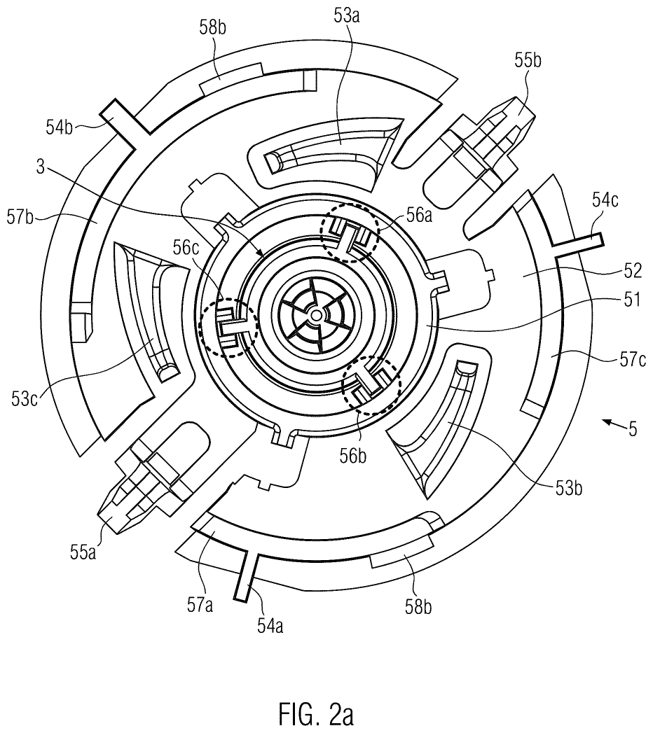

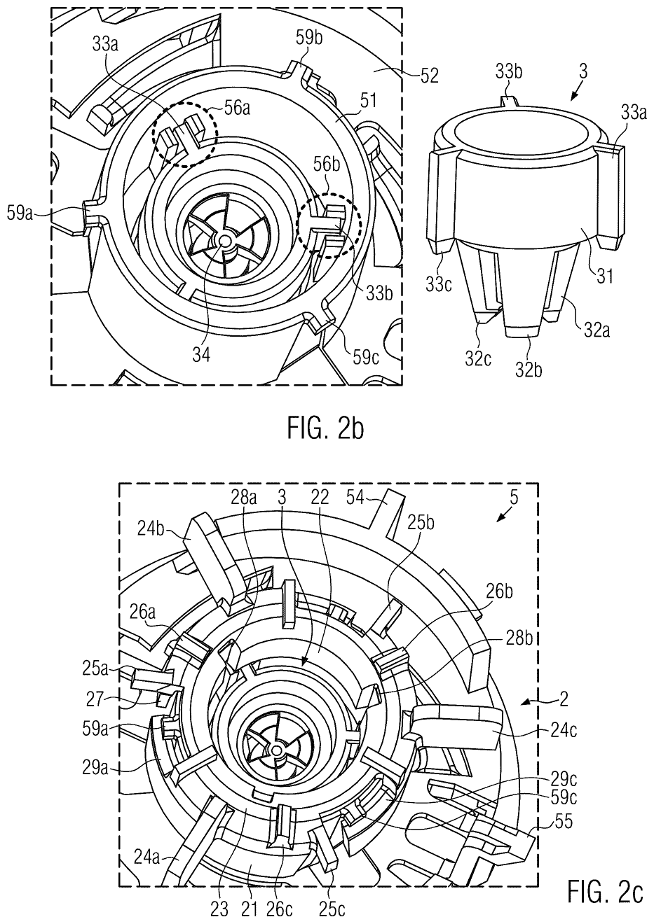

In FIG. 2a, details of the inserter sleeve 5 and the retractor 3 are shown. The view in FIG. 2a is provided onto the inserter sleeve 5 in the direction from the distal end 17 towards the proximal end 18. The inserter sleeve 5 may be provided with an inner sleeve portion 51 in which the retractor 3 is provided. The inserter sleeve 5 may provide one or more guide portions such as three or more guide portions 56a, 56b, 56c which in FIG. 2 are exemplarily shown as a negative rail portion which provide a slot. The slot may also be provided without two negative rails but by a depression into a portion of the inserter sleeve 5 such as a depression in the inner sleeve portion 51. The guide portions may provide a part of the movement path of the retractor. Another part of the movement path of the retractor maybe provided by guide portions of the retractor control element.

The inserter sleeve 5 may, furthermore, be provided with an outer sleeve member 57 having several outer sleeve member parts such as parts 57a, 57b and 57c which can be called wall portions of the inserter sleeve 5. The outer sleeve member 57 is not an entirely closed sleeve but may comprise several openings or interruptions. The wall portions 57 may be provided on a cylindrical arrangement. The inserter sleeve 5 may furthermore have a disc shaped element 52, which may connect the different functional portions of the inserter sleeve. It may, for example, connect the inner sleeve portion 51 to the outer sleeve portion(s) 57. The disc-shaped element 52 may provide support for beams 53 which may be provided by the inserter sleeve 5. The inserter sleeve 5 may comprise one or more, two or more or three or more beams 53a, 53b, 53c which allow to elastically bias the cap 1 towards the distal end 17 as will be explained further below.

The inserter sleeve 5 may further comprise one or more such as two or more or three or more guide portions 54a, 54b, 54c which cooperate with guide portions of the guide sleeve 6 as will be explained further below. The guide portions 54a, 54b and 54c may be provided at equal distance with respect to each other (or at least two of the distances between the three guide portions 54a, 54b, 54c may be equal), as this provides for a safe way to avoid blocking of the movement of the inserter sleeve 5 with respect to the guide sleeve 6. They may, for example, be provided by an equal circumferential separation.

The disc-shaped element 52 may have an essentially circular outer perimeter.

The inserter sleeve 5 may, furthermore, provide device retention hooks 55a and 55b. There may also be more than two device retention hooks 55, while in FIG. 2a only two device retention hooks 55a and 55b are shown. The device retention hooks 55 hold the medical device 7 prior and during the insertion process. The device retention hook(s) are an example of a holder for the medical device 7. It may release the medical device 7 after or at the end of the insertion process.

The inserter sleeve 5 may further comprise one or more, such as two or more or three or more openings (without reference signs) which are provided for the separation limiting rib 15 to reach through the inserter sleeve 5 and engage with the inserter sleeve 5 from the back side shown in FIG. 2a which is the proximal end 18 side.

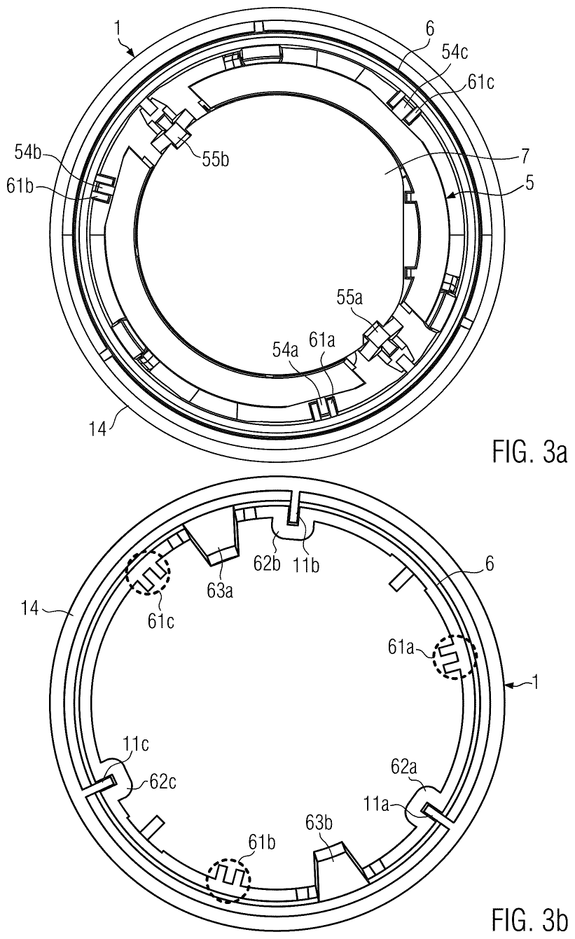

In FIG. 2b the retractor 3 is shown. The retractor 3 has a retractor body 31 which may be provided with an essentially cylindrical form. The retractor body 31 may be provided as a sleeve. On its outside, the retractor 3 may have guide portions 33 such as two or more or three or more guide portions 33a, 33b, 33c. The guide portions 33 may have an end (proximal end or lower end in FIG. 3) which is tapered which allows an easy assembly of the retractor 3 in the inserter sleeve 5. On its proximal end the retractor 3 may have hooks 32. In FIG. 2b the retractor 3 is provided with three hooks but there may be only two hooks or four or more hooks as well. The hooks 32a, 32b and 32c may be provided in order to engage with a portion 8 provided with the medical device 7 which is to be retracted. The retractor 3 may further be provided with a center portion 34 which prevents a movement of a portion 8 that is to be retracted towards the distal end 17. As further shown in FIG. 2b, the guide portions 33 may cooperate with the guide portions 56 of the inserter sleeve 5 in order to provide for a linear (only) movement of the retractor 3 with respect to the inserter sleeve 5.

As further shown in FIG. 2b, the inserter sleeve 5 may further be provided with coupling protrusions 59 such as two or more or three or more coupling protrusions 59a, 59b, 59c. These coupling protrusions 59 are provided in order to define a (rotational or purely rotational) movement of the retractor control element 2 with respect to the inserter sleeve 5. The coupling protrusions 59a, 59b, 59c may be provided on the outer side of the inner sleeve portion 51.

In FIG. 2c, a 3D view of the retractor control element 2 is shown in combination with the retractor and the inserter sleeve 5. The main body of the retractor control element 2 may be provided with an outer sleeve member 21 and an inner sleeve member 22 which may be connected by a connection member 23. The connection member 23 may be provided at the distal end of the outer sleeve member 21 and the inner sleeve member 22.

The retractor control element 2 may be provided on its outside with protruding portions 24, which may be used to provide a force to the retractor control element 2 which rotates the retractor control element 2. The protruding portion 24 may be provided at the outside of the retractor control element 2, which allows for a finer dosing and control of the force that creates the rotational movement but they may also be provided on its distal end or on its inner side. The retractor control element 2 has, essentially, a ring shaped body. This ring shaped body may comprise the outer sleeve member 21, the inner sleeve member 22 and the connection member 23.

In the embodiment shown in FIG. 2c, the retractor control element 2 has three protruding portions 24a, 24b and 24c. It may also have or also be possible to provide only two or four or more protruding portions 24. In the embodiment shown in FIG. 2c, the protruding portions 24a, 24b, 24c are provided with equal distances with respect to each other. This allows to ensure a smooth rotational movement without blocking of the retractor control element 2 during its rotational movement. It may also be that only two of the distances between different protruding portions 24 are equal.

The retractor control element 2, furthermore, may be provided with one, two, three or more or locking portions 25. These locking portions 25 are provided on the outside of the main body but they may also be provided on the distal end 23 or on the inner side of the main body. The locking portions 25 can prevent a rotation of the retractor control element 2 in combination with the cap, as will be explained further below.

The retractor control element 2 may be provided with one, two, three or more blocking protrusions 26. In FIG. 2c, three of such blocking protrusions 26, as shown. These protrusions 26a, 26b, 26c are shown on the distal end of the main body and in particular, on the connection member 23. They may also be provided on the outer side such as on the outer sleeve member 21 or on the inside such as on the inner sleeve member 22.

The retractor control element 2 may be provided to sit rotationally slidable on the inserter sleeve 5 and in particular on a dedicated portion of the inserter sleeve 5. In particular the retractor control element 2 may be provided to sit rotationally slidable on the inner sleeve portion 51 of the inserter sleeve 5. In this arrangement, the inner sleeve member 22 of the retractor control element 2 is provided on the inside of the inner sleeve portion 51 of the inserter sleeve 5, the connection member 23 is provided on the distal end of the inner sleeve portion 51 of the inserter sleeve 5 and the outer sleeve member 21 is provided outside of the inner sleeve portion 51 of the inserter sleeve 5. Thereby, the good support and guiding of the retractor control element 2 is provided for a rotational movement.

In order to avoid a separation of the retractor control element 2 from the inserter sleeve 5 by a movement of the retractor control element 2 towards the distal end 17 of the insertion device, the coupling protrusions 59 are provided on the inserter sleeve 5 which prevent a movement of the retractor control element 2 towards the distal end 17. In FIGS. 2b and 2c these coupling protrusions 59a, 59b and 59c are provided on the outside of the inner sleeve 51 but they may also be provided on the inside, thereof.

Co-operating with the coupling protrusions 59, there may be provided slots 29 in the retractor control element 2 which accommodate the coupling protrusions 59. The bottom of the slots 29 thereby are in contact with the bottoms of the coupling protrusions 59. The retractor control element 2 may, furthermore, be provided with an assembly slot 27 which connects the slots 29 with the proximal end of the retractor control element 2. Thereby, the protrusions 59 of the inserter sleeve 5 may be brought into the slots 29 during assembly of the insertion device.

The bottom surface(s) of the retractor control element 2 may be provided to come in contact with guide portions of the retractor 3. This may refer in particular to the guide portions 33 as explained above. The bottom surface(s) of the retractor control element 2 is/are a surface/surfaces facing towards the proximal end 18 of the insertion device. As long as the guide portions 33 of the retractor 3 come in contact with the bottom surface(s) of the retractor control element 2, the retractor 3 is blocked from moving in the direction towards the distal end 17.

The retractor control element 2, furthermore, has guide portions 28 which do allow a movement of retractor 3 towards the distal end 17 as soon as these guide portions 28 are aligned with the guide portions 33 of the retractor 3. As shown in FIG. 2c, the retractor control element 2 is in a position where the guide portions 28 are not in alignment with the guide portion 33 and the retractor 3 is blocked from moving towards the distal end 17.

The guide portions 28 (28a, 28b, 28c) may be provided as one, two, three or more guide portions. If more than one guide portion 28 is provided then those guide portions or some of the guide portions 28 are preferably separated by an equal distance with respect to each other. This avoids an unintentional blocking of the retractor 3 while moving relative to the contractor control element 2.

The retractor control element 2 may be arranged such that it can be rotated into a position in which the guide portions 28 are aligned with the guide portions 56 of the inserter sleeve. Thereby, the movement of the retractor towards the distal end 17 can be enabled. This is done by freeing the moving path of retractor 3 in the direction of the distal end 17. The movement path is in this case provided by the combination of the aligned guide portions 28 of the retractor control element and the guide portions 56 of the inserter sleeve.

In FIG. 3a a view onto the proximal end 18 of the insertion device in the direction towards the distal end 17 is shown. The most outer part is the proximal end of the cap 1. Inside of the cap 1 is shown the guide sleeve 6 with its proximal end surface. Guide portions 61 of the guide sleeve are shown which cooperate with guide portions 54 of the inserter sleeve. In the embodiment shown in FIG. 3a, there are three pairs of guide portions 54, 61, namely, guide portions 54a, 54b and 54c co-operating with guide portions 61a, 61b, 61c, respectively. The guide portions are arranged at equal distances with each other. As can be seen, the insertion device may have an essentially circular cross section.

In FIG. 3a, the bottom surface of the medical device 7 is shown. This surface is supposed to come in contact with the skin 9 of the subject. On this bottom surface of the medical device 7, e.g., an adhesive may be provided which adheres the medical device 7 to the skin 9 of the subject.

In the middle of the medical device 7, a needle and/or a sensor may be provided that need to be inserted through the skin 9 of the subject. In FIG. 3a, it is shown that the inserter sleeve 5 may be provided with device retention hooks 55. In the embodiment in FIG. 3a, two of such device retention hooks 55, namely, 55a, 55b are shown. However, it is also possible to provide only one or three or more device retention hooks 55. During the insertion process, the device retention hooks 55 move outwardly, thereby freeing the medical device 7 from its position within the insertion device. The two device retention hooks may be provided opposite to each other with respect to the center of the insertion device.

FIG. 3b shows a view from the relation of the cap 1 with respect to the guide sleeve 6. The cap 1 is provided on its inside, in particular on the inside of the housing portion 14, with guide portions 11. As shown in the embodiment in FIG. 3b, there are provided three such guide portions 11, namely, 11a, 11b and 11c. The guide portions 11 can cooperate with corresponding guide portions 62 provided by the guide sleeves 6. In the embodiment shown in FIG. 3b, the guide portion 11 is provided as a positive rail and the guide portion 62 of the guide sleeve 6 as a negative rail or as a slot. The inverse arrangement is possible as well in which the guide portions 62 of the guide sleeve 6 are a positive rail and the guide portions 11 of the cap are a negative rail/slot.

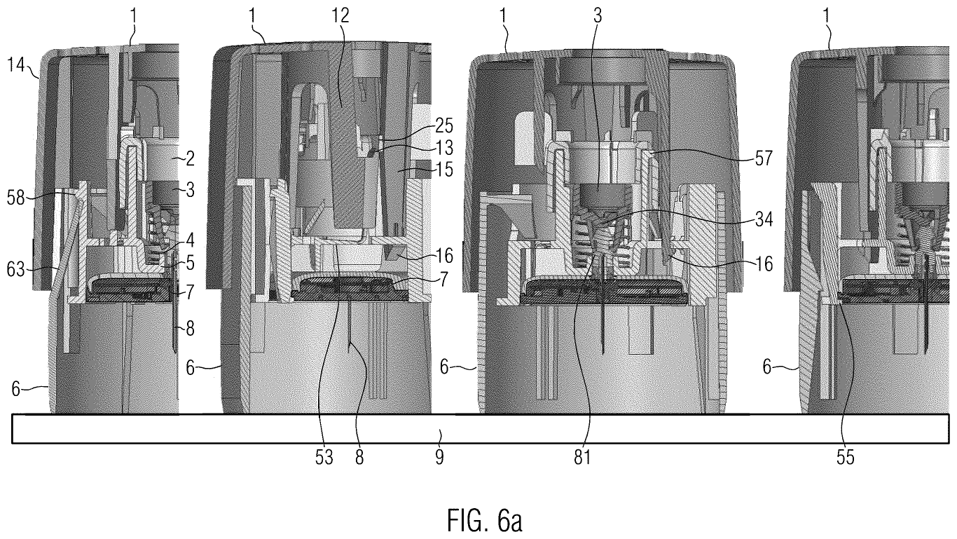

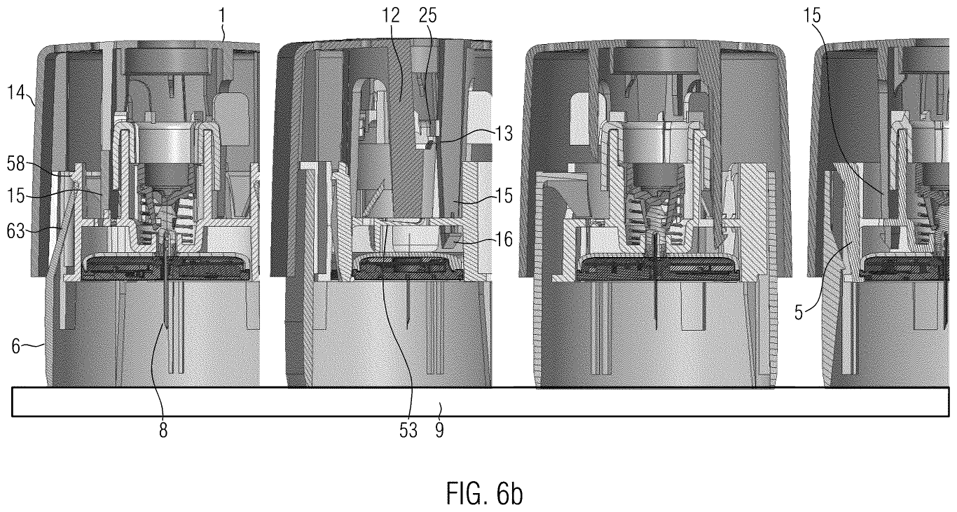

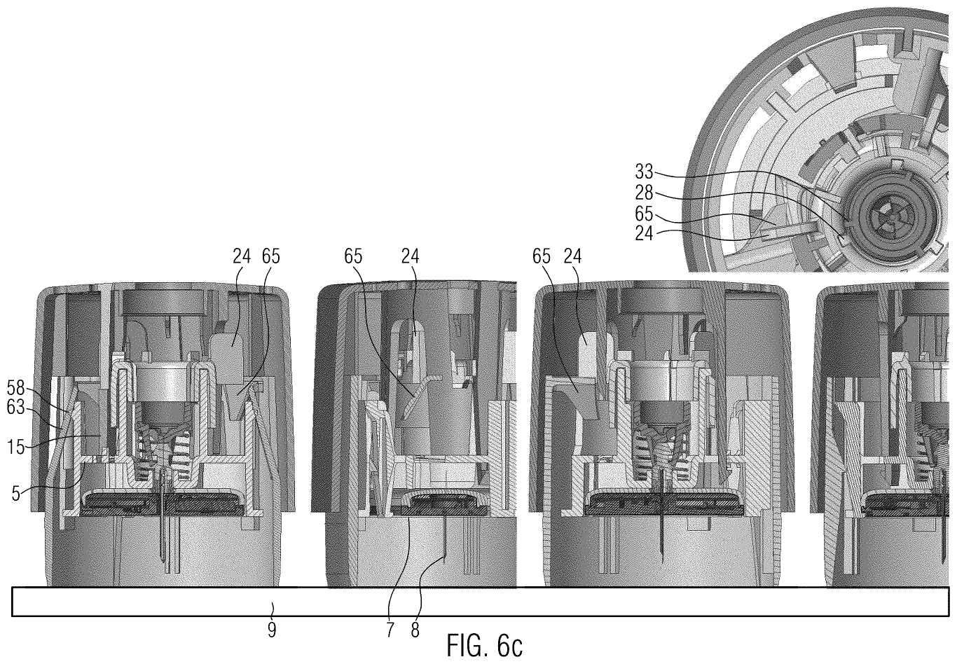

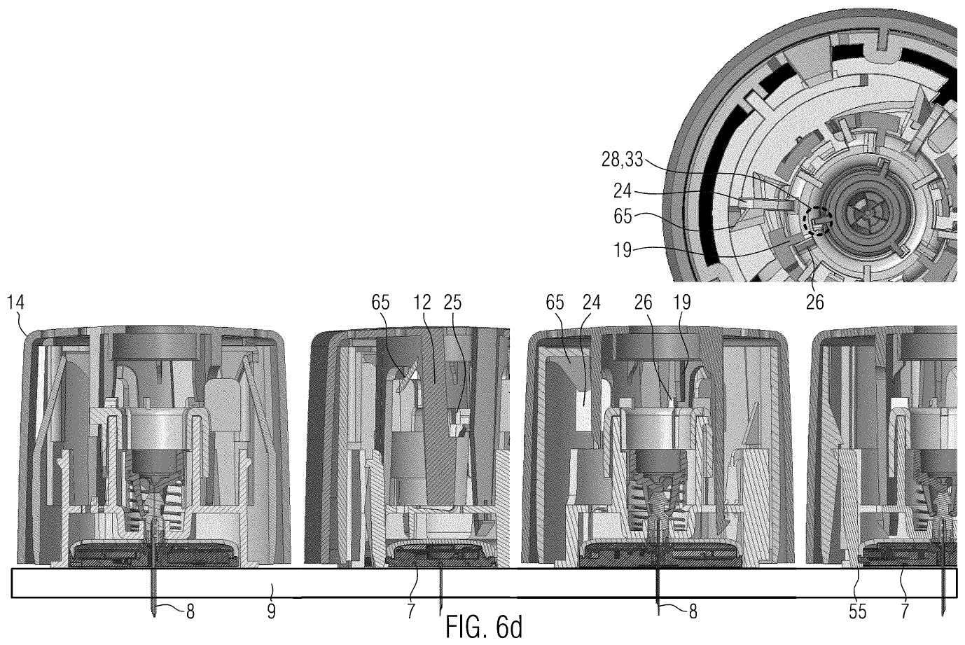

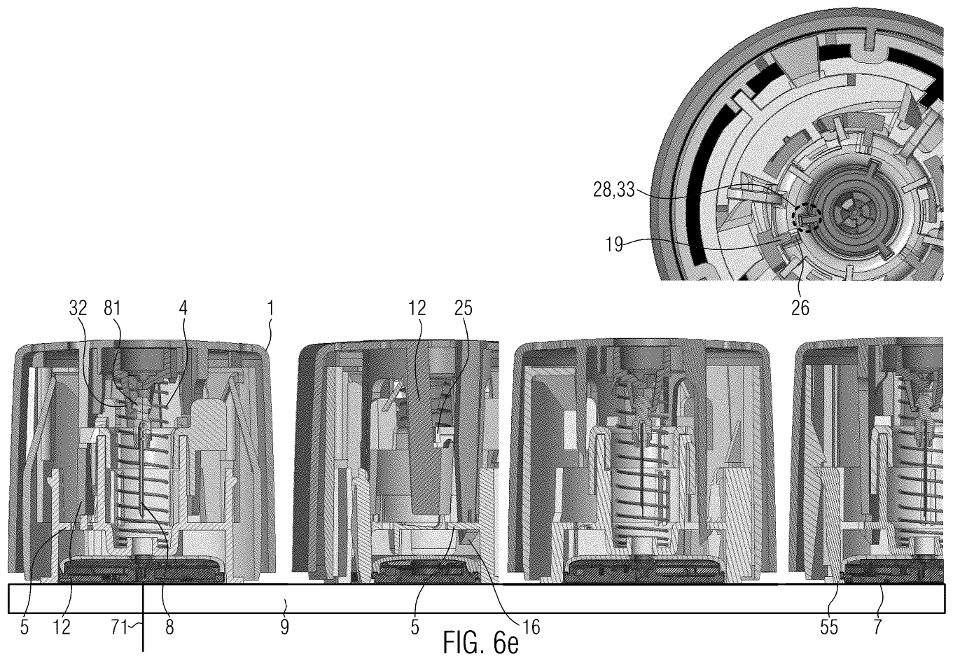

Furthermore, shown in FIG. 3b are guide portions 61 on the inside of the guide sleeve. Those guide portions are already discussed in relation with FIG. 3a. In FIG. 3b, it is shown that the guide sleeve 6 may comprise one or more retainer beams 63. Such retainer beams 63 maintain the inserter sleeve 5 in a distal position unless a certain amount of force is applied to the cap 1 towards the proximal end 18. This will be explained in further detail in relation to FIG. 6.

As can be seen in FIG. 3b, the guide sleeve 6 can have an essentially circular cross section.

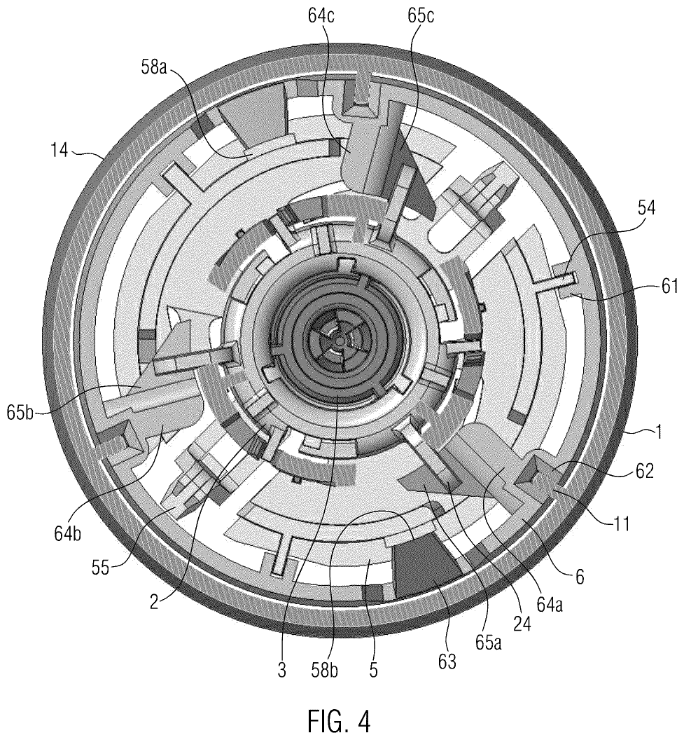

FIG. 4 shows a view from the distal end 17 towards the proximal end 18 of the insertion device with the cut just above the retractor control element 2. In this view, ramps 65 are shown which can cooperate with protruding portions 24 as will be discussed further below.

In the view in FIG. 4, it is furthermore shown that inner portions of the cap 1 cooperate with parts of the retractor control element 2 which limit, or allow, or block, or free a rotational movement of the retractor control element as will be discussed further in relation to FIG. 6.

As can be seen in FIG. 4, the ramps 65 can be provided as an inner part of the guide sleeve 6 and each ramp can be connected to remaining parts of the guide sleeve 6 by a ramp support 64.

As shown in FIG. 4, there can be three ramps 65, namely, 65a, 65b, 65c. It is also possible to have fewer than three, such as one or two ramps, or more than three ramps. As the ramps 65 cooperate with the protruding portions 24 of the retractor control element 2 the number of such protruding portions 24 of the retractor control element 2 and the number of ramps 65 of the guide sleeve 6 may be provided in the same number. In the embodiments shown in FIG. 4, there are three ramps 65 co-operating with three protruding portions 24.

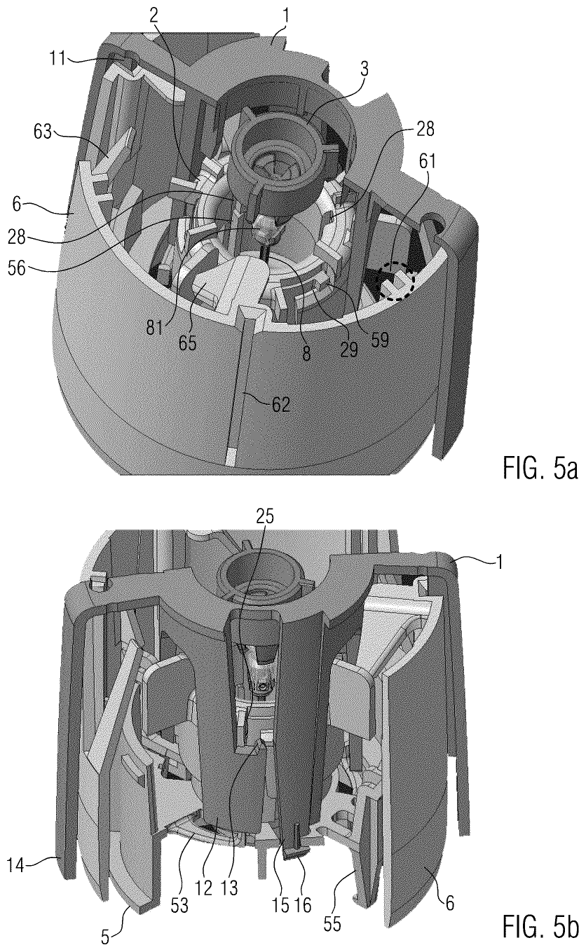

FIG. 5a shows a three-dimensional view into the insertion device with parts of it removed in order to make interior components visible.

As can be seen in FIG. 5a on the outside of the essentially circumferential wall portion of the guide sleeve 6 negative rails or slots 62 are provided which guide the guide portions 11 of the cap 1. Furthermore, the guide portions 61 on the inside of the guide sleeve 6 are shown. Furthermore, in FIG. 5a it is shown that ramps 65 may be provided as part of the guide sleeve 6 and facing towards the inner part of the insertion device.

In FIG. 5a, the retractor 3 is shown in a position in which it has moved towards the distal end 17 of the insertion device, as the retractor 3 is shown above the retractor control element 2. In FIG. 5a the spring 4 which has pushed the retractor 3 towards the distal end 17 is not shown for clarity.

In FIG. 5a, it is shown that the retractor 3 can be in engagement with the head of a portion that is to be retracted—here head 81 of the needle 8.

In the configuration shown in FIG. 5a, the guide portions 28 of the retractor control element 2 are shown in alignment with the guide portions 56 of the inserter sleeve 5 that define (at least a portion of) the movement path of the retractor 3. In that position, the protrusion 59 is at the end of the slot 29 of the retractor control element 2, thereby limiting the rotational movement of the retractor control element beyond the alignment of the guide portions 28 with those guide portions 56 of the inserter sleeve 5.

FIG. 5b provides another view into the insertion device with other parts removed in order to provide visibility of interior portions. In FIG. 5b in particular, it is shown that the cap 1 may be provided with a pushing portion 12 which extends from the top surface of the cap 1 at its distal end towards the proximal end of the insertion device. This pushing portion 12 is arranged such that it can come into contact with beams 53 of the inserter sleeve 5. Furthermore, the pushing portion 12 can also come into contact with other parts of the inserter sleeve 5, allowing to push the inserter sleeve 5 towards the proximal end 18 of the device during the insertion process. As shown in FIG. 5b, the cap 1 may further be provided with an engagement portion 13 which interacts with the locking portion 25 provided on the retractor control element 2. The engagement portion 13 may prevent a rotation of the retractor control element 2. This may be the case, at least as long as the cap 1 is in its most distal position.

In FIG. 5b it is furthermore shown that the cap 1 may comprise a separation limiting rib 15 which at its lower end may be provided with a separation limiting hook 16. This separation limiting rib 15 can extend from the top surface of the cap 1 at its distal end towards the proximal end 18 of the insertion device. It thereby reaches through an opening in the inserter sleeve 5, such that the separation limiting hook 16 can come into contact with the surface of the inserter sleeve 5 facing towards the proximal end 18 of the insertion device. In this way, the uncontrolled separation of the cap 1 into the distal direction 17 away from the inserter sleeve 5 is prevented.

The pushing portion 12 in co-operation with the separation limiting rib 15 and the separation limiting hook 16 may provide or define a limited movement range of the cap 1 with respect to the inserter sleeve 5, the movement of those two portions relative to each other may be limited to a range between 0.5 and 2 mm, for example.

In the configuration shown in FIG. 5b, the locking portion 25 is not in contact with the engagement portion 13 but, as the retractor control element 2 has already been rotated, the locking portion 25 now comes into contact with a portion of the cap 1 such as the pushing portion 12 or the separation limiting rib 15. Thereby, the rotation of the retractor control element 2 beyond the position in which the guide portions of the retractor control element and of the inserter sleeve 5 are aligned is prevented.

In FIGS. 6a to 6e, different sections through the insertion device are shown, wherein in each FIGS. 6a to 6e, the different shown sections correspond to the same state in which the insertion device is shown.

FIG. 6a shows a state of the insertion device in which the insertion device is provided with the medical device 7 to be inserted. The medical device 7 is shown at the proximal end of the inserter sleeve 5 and it is retained by device retention hooks 55 (shown in FIG. 6a, most right view). The medical device 7 is provided together with a needle 8. The needle 8 is to be inserted through the skin 9 of a subject. Together with the needle 8 may be provided an analyte sensor which is to be inserted through skin 9 of a subject.

The guide sleeve 6 is provided with its proximal end portion on the skin 9 of the subject.

In the configuration shown in FIG. 6a the inserter sleeve 5 is shown to be able to rest with its blocking portion 58 on retainer beam 63 of the guide sleeve. The spring 4 (elastic element) is in its preloaded state and compressed against its spring force. The spring 4 may rest with its proximal end in a ring shaped depression of the inserter sleeve 5. The distal end of the elastic element 4 is in contact with the retractor 3 and biases the retractor 3 in the distal direction (upward in FIGS. 6).

In the orientation shown in FIG. 6, the lower end of the insertion device corresponds to its proximal end 18 which is the end that is in contact with the skin 9 and the distal end 17 refers to the upper end shown in FIG. 6a.

As can be seen in FIG. 6a, the retractor 3 is in an engaged relation with the head 81 of a needle 8. This engagement relation can be achieved by inserting the medical device together with the head 81 into the inserter sleeve 5. The medical device 7 thereby opens the retention hooks 55 and as they snap back into a closed position they retain the medical device 7 in the position shown in FIG. 6a. When adding the medical device 7 to the insertion device the head 81 of the needle 8 also opens the hooks 32, and as the head 81 has come into contact with the center portion 34 of the retractor, the hooks 32 snap back inwardly and thereby retain the head 81.

As shown in FIG. 6a in the second section from the left, cap 1 is pushed upwards by the pushing portion 12 which rests on beam 53. This beam 53 is slightly elastic and elastically pushes the cap 1 into its most distal (upward in FIG. 6a) position.

The separation limiting hook 16 may be in contact with a lower surface of the inserter sleeve 5 and thereby prevents a further upward movement of the cap 1 in relation to the inserter sleeve 5.

As furthermore shown in the second intersection from the left, the locking portion 25 is in contact with the engagement portion 13 and thereby prevents rotation of the retractor control element 2. The position shown in FIG. 6a may be the starting position with respect to the rotational movement of the retractor control element 2. When viewed from the top, i.e., in the direction from the distal end 17 towards the proximal end 18, the rotation of the retractor control element 2 is in the clockwise direction as will be discussed further below.

The right most intersection shows that the inserter sleeve 5 may be provided with a device retention hook 55 which retains the medical device 7 in its position as shown in FIG. 6a.

As long as the cap 1 is in its upmost position (most distal position) shown in FIG. 6a, the contact of the locking portion 25 with the engagement portion 13, prevents the rotation of the retractor control element 2 also in case the device as shown in FIG. 6a falls down and drops on a surface such as a table or a floor. The engagement portion 13 may allow the rotation of the retractor control element 2 only once the cap 1 is moved into a lower position as will be discussed in the following.

In FIG. 6b the situation is shown in which some force or some pressure has been applied to the top surface of the cap 1. The inserter sleeve 5 still remains in its original position as the blocking portion 58 still rests on the retainer beam 63. However, as can be seen in FIG. 6b, the cap 1 may now be in contact with the inserter sleeve 5 such that the force applied to the cap 1 translates into an equal force onto the inserter sleeve 5. In the situation shown in FIG. 6b it can be seen that the pushing portion 12 can have slightly bent the beams 53 against the small force (smaller than the force provided to press on the cap 1) provided by the beams 53.

Further, it can be seen that the separation limiting hooks 16 has lost contact with the lower surface of the inserter sleeve 5 as the cap 1 has travelled down a little bit with respect to the inserter sleeve 5. This can be seen in particular in the three most right sections shown in FIG. 6b.

As can be seen in FIG. 6b, as the cap 1 has moved downward relative to the retractor control element 2, the locking portion 25 is no longer in contact with the engagement portion 13 of the cap and thereby the rotational movement of the retractor control element 2 is made possible.

After having applied further pressure on the cap 1, the blocking portions 58 no longer rest on the retainer beams 63 but have passed below them (see FIG. 6c). The force on the cap 1 may have translated to a force onto the inserter sleeve 5 by, e.g., a contact of a pushing portion 12 of the cap 1 with the inserter sleeve 5. As can be seen in particular in the second section from the left the protruding portion 24 now comes into contact with the ramp 65 of the inserter sleeve 5. As the surface of the ramp 65 is inclined (With respect to a direction perpendicular to skin onto which the insertions device can be placed and/or with respect to a longitudinal axis of the insertion device and/or with respect to the direction of the linear movement of the cap towards proximal end 18), the downward movement of the retractor control element 2 translates into rotational movement of the retractor control element 2. As can be seen in the upper inset in FIG. 6c, the guide portions 28 of the retractor control element 2 are not yet in alignment with the guide portions 33 of the retractor 3.

As the inserter sleeve 5, the elastic element (spring) 4, the retractor 3 and the retractor control element 2 together with the cap 1 travel further down and make a relative movement with respect to the guide sleeve 6, the protruding portions 24 rotate further in sliding contact with the ramp 65 and the rotation of the retractor control element 2 progresses as the cap 1 of the retractor control element 2, the elastic element (spring) 4 and the inserter sleeve 5 travel further down in FIG. 6.

In FIG. 6d, a state is shown in which the cap 1 has reached its lowest most position. The cap 1 now can rest on the guide sleeve 6. The needle 8 has penetrated the skin 9 of the subject and the bottom surface of the medical device 7 has come into contact with the skin 9 of the subject. In case that this bottom surface is provided with an adhesive the portion of the medical device 7 may adhere to the skin 9 of the subject. In that position the guide sleeve interacts with a portion of the inserter sleeve 5 which opens the device retention hook 55 (this is not shown in FIG. 6d).

As can be seen in the third intersection from the left, the protruding portions 24 have passed the lower end of the ramps 65 and the ramps (such as the lower end of the ramps) provide a slightly elastic force onto the protruding portions 24 into the rotational direction of the retractor control element 2.

In the situation in FIG. 6d it is shown that blocking protrusion 26 of the retractor control element 2 may come in contact with a blocking portion 19 of the cap 1 which prevents the rotational movement of the retractor control element 2 to complete. This can also be seen in the upper inset in FIGS. 6d in which it can be seen that the guide portions 28 are not yet in full alignment with the guide portions 33 of the retractor 3.

In FIG. 6e the situation is shown in which the cap 1 is slightly moved upwards (in the distal direction). In this configuration the blocking portion 19 may lose contact with the blocking protrusion 26 so that the retractor control element 2 may complete its rotational movement into a position as shown in the upper inset as shown in FIGS. 6e. In this position as shown the guide portions 28 may come in full alignment with the guide portions 33 of the retractor 3 such that by the force of the elastic element 4 the retractor now travels in the distal direction (upwardly in FIG. 6d). This movement may move the retractor 3 through the retractor control element 2.

The movement of the retractor 3 caused by the elastic element 4 may push the retractor 3 through the retractor control element 2 to an extent that the retractor 3 loses contact with the retractor control element 2. The retractor thereby may leave the movement path that may be considered to be defined at least in part by the guide portions of the retractor control element 2.

The retractor control element 2 completes its rotation due to the force exerted by the lower end portions of the ramps 65 onto the protruding portions 24.

The upward movement of the cap 1 in FIG. 6e is limited by the separation limiting hook 16 coming into an engagement with the inserter sleeve 5.

The needle 8 is retracted into a position such that a contact of the needle 8 with a user can be avoided by being retracted within the insertion device.

After the needle 8 being retracted, a sensor 71 of the medical device 1 may remain inserted through the skin 9 of the subject. The needle 8 is no longer in contact with the medical device.

The insertion device 1 . . . 6 may then be lifted from the medical device 7 which may stay in contact with the skin 9 of the subject and the insertion process is completed.

While exemplary embodiments have been disclosed hereinabove, the present invention is not limited to the disclosed embodiments. Instead, this application is intended to cover any variations, uses, or adaptations of this disclosure using its general principles. Further, this application is intended to cover such departures from the present disclosure as come within known or customary practice in the art to which this invention pertains and which fall within the limits of the appended claims.

### LIST OF REFERENCE SIGNS

1 Cap
   11 guide portions, rail
   12 pushing portion
   13 engagement portion
   14 housing portion
   15 separation limiting rib
   16 separation limiting hook
   17 distal end
   18 proximal end
   19 blocking portion
2 Retractor control element
   21 outer sleeve member
   22 inner sleeve member
   23 connection member
   24 protruding portion
   25 locking portion
   26 blocking protrusion
   27 assembly slot
   28 guide portion, negative rail
   29 slot
3 Retractor
   31 retractor body
   32 hooks
   33 guide portions, positive rail
   34 centre portion
4 Elastic element
5 Inserter sleeve
   51 inner sleeve portion
   52 disk-shaped element
   53 beam
   54 guide portion
   55 device retention hook
   56 guide portion, negative rail
   57 outer sleeve member
   58 blocking portion
   59 coupling protrusion
6 Guide sleeve
   61 guide portion, negative rail
   62 guide portion, negative rail
   63 retainer beam
   64 ramp support
   65 ramp
7 Medial device
   71 sensor
8 Needle
   81 head of needle
9 Skin

What is claimed is:

1. An insertion device for at least partially inserting a medical device through the skin of a subject, the insertion device comprising:
a proximal end and a distal end opposite the proximal end;
a guide sleeve at the proximal end and configured to contact the skin for an insertion process;
a cap at the distal end, the cap configured to be pressed to carry out at least part of the insertion process while moving in the direction of the proximal end;
an inserter sleeve at least partially within the guide sleeve and/or the cap and having a holder for the medical device, the inserter sleeve being moveable with respect to the guide sleeve;
a retractor configured to retract a portion provided with the medical device, the retractor being moveable along a movement path;
an elastic element configured to bias the retractor towards the distal end along the movement path; and
a retractor control that controls the movement of the retractor by blocking or freeing the movement of the retractor along the movement path, the retractor control being rotatable with respect to the retractor.

2. The insertion device of claim 1, wherein the retractor control is rotatable between a first position in which it blocks the movement of the retractor along the movement path and a second position in which it frees the movement of the retractor along the movement path.

3. The insertion device of claim 1, wherein the retractor control is rotatable by a portion of the guide sleeve that contacts a protruding portion of the retractor control upon a relative movement of the retractor control with respect to the guide sleeve in the direction of the proximal end.

4. The insertion device of claim 1, wherein the movement of the retractor along the movement path is confined to a linear movement in the direction of the proximal end to the distal end or vice versa, the confinement to the linear movement optionally being provided by at least a pair of guide portions, the pair of guide portions comprising a rail and a cooperating negative rail, one of the two guide portions being provided at the inserter sleeve and the other guide portion provided at the retractor, wherein, optionally, two or more said pairs of guide portions are provided.

5. The insertion device of claim 4, wherein the retractor control has at least one guide that can be brought into alignment with the guide portion of the inserter sleeve in order to free the movement of the retractor along a portion of the movement path and block the movement of the retractor along the portion of the movement path when the guide of the retractor control is not in said alignment.

6. The insertion device of claim 1, wherein the retractor control has a blocking protrusion configured to interact with a blocking portion of the cap, wherein contact of the blocking protrusion with the blocking portion prevents rotation of the retractor control into a position in which it frees the movement path of the retractor.

7. The insertion device of claim 6, wherein the contact of the blocking protrusion of the retractor control and the blocking portion of the cap is possible in a position of the cap in its most proximal position upon being pressed.

8. The insertion device of claim 1, wherein the retractor control comprises a lock that is in contact with an engagement portion of the cap, wherein the lock prevents rotation of the retractor control when the cap is positioned at its most distal position.

9. The insertion device of claim 1, wherein the retractor control is rotatably mounted on the inserter sleeve.

10. The insertion device of claim 1, wherein the cap is configured to move linearly relative to the guide sleeve, the linear movement being guided by a pair of guide portions, the pair of guide portions having a first guide portion in the cap that cooperates with a second guide portion on the guide sleeve, wherein the insertion device optionally is provided with at least three said pairs of guide portions, wherein the guide portions further optionally are provided equally spaced to each other.

11. The insertion device of claim 1, wherein the cap has one or more pushing portions configured to push the inserter sleeve towards the proximal end and optionally with separation limiting means for limiting a separation of the cap away from the inserter sleeve towards the distal end.

12. The insertion device of claim 1, wherein the cap, the inserter sleeve, the retractor, the retractor control and the elastic element are movable from a distal position to a proximal position together at least along a portion of the movement of the cap, optionally without a relative movement relative to each other.

13. The insertion device of claim 1, wherein the elastic element is provided in a preloaded state acting between the retractor and the inserter sleeve.

14. An insertion system, comprising:
the insertion device of claim 1; and
a medical device configured to be inserted with the insertion device.

15. A method of using the insertion device of claim 1, comprising:
applying the insertion device to the skin of a subject;
pressing the cap towards the skin of the subject;
translating the linear movement of the cap relative to the guide sleeve into a rotational movement of the retractor control;
the retractor control freeing a movement path of the retractor towards the cap by rotation of the retractor control, optionally after the cap having slightly retracted in a direction away from the skin of the subject.
